(19) United States
(12) Patent Application Publication
(10) Pub. No.: US 2014/0277118 A1
(43) Pub. Date: Sep. 18, 2014

(54) CLOSURE DEVICE FOR CLOSING OPENED BLOOD VESSELS

(71) Applicant: VOSTRA-MED AG, Cham (CH)

(72) Inventors: Ernst-Diethelm Harren, Weinfelden (CH); Felix Banthien, Geisingen (DE)

(21) Appl. No.: 14/350,397

(22) PCT Filed: Oct. 17, 2012
(86) PCT No.: PCT/CH2012/000237

§ 371 Date: Apr. 8, 2014

(30) Foreign Application Priority Data
Oct. 18, 2011 (DE) 20 2011 106 809.2
Oct. 18, 2011 (EP) 11405339.9

(51) Int. Cl. A61B 17/00

(52) U.S. Cl. CPC A61B 17/0057
USPC 606/213

(57) ABSTRACT

A closure device (1) which can be adhered in a defined position for closing opened blood vessels has a closure element (2) and a pressure body (3), said closure element (2) being made of a material with elastic properties. The closure device (1) further contains a plurality of retaining strips (4) which adjoin distal ends of the closure element (2). The retaining strips (4) are provided with a reinforcing layer (5) at least in some regions, said reinforcing layer being made of a material with non-elastic properties. The closure device also contains at least one indicator element (8) which is connected to the closure element (2). The advantages consists in a simple design of the adhesive closure device (1) and in that the closure device can be handled easily even by laypeople.

The invention relates to a closure device for closing opened blood vessels according to claim 1.

The invention relates in particular to a closure device which can be adhered in a defined position for closing opened blood vessels, comprising a pressure body which is suitable for coming into contact with a wound.

In this area of medical applications, in particular however in the puncturing of vessels, there is still a requirement to make available a closure device for closing opened blood vessels, in the following also referred to as a vessel closure, which can also be handled easily by a medical layperson and with which not only the subsequent discharge or loss of blood into the open space outside of the body, but also the discharge of blood into the tissue (haematoma formation) is prevented, at the same time, however, the circulation of blood, essential to life, within the body not being hindered after having applied the closure device.

In the conventional manner the closure of puncture points of a blood vessel takes place such that after intervening at the vessel puncture point a pressure is exerted. Due to the risk of haematoma formation the pressure must not be too small, but due to the risk of stopping circulation in the respective part of the body it must not be too great either. There are therefore solutions in which one attempts to establish a sufficient counter-pressure, i.e. a counter-pressure corresponding to the blood pressure, by means of an external blood chamber or an external pressure chamber filling with the discharged blood and in this way a closure device acting as a pressure body. Additional mechanical pressure bodies are partially also used as back-up.

An example of this type of closure device is described in DE 44 29 230. This publication shows a puncture closure that can be adhered, comprising a pressure chamber and an extendable pressure wall. This puncture closure is already adhered before puncturing the vessels. The pressure wall and the pressure chamber are pierced upon puncturing.

However, it is a disadvantage of the puncture closure that can be adhered according to DE 44 29 230 that it has a relatively complicated structure due to the ever-present pressure chamber. Moreover, it is not very easy to handle because the vessels to be punctured must always be punctured by the closure element and the extendable pressure wall of the pressure chamber.

A further example of a vessel closure is disclosed in WO 2006/042430 A1. This vessel closure contains a closure layer and a pressure body which are made in one piece of an extendable material. Furthermore, a carrier layer is provided which is extensively connected to the closure layer by means of an adhesive layer. By means of a skin adhesive layer, which is in turn connected to the carrier layer, the vessel closure can be adhered to the patient's skin in the region of the puncture opening. The problem here is that the extensive, multi-layered structure causes high costs. A further problem is that the pressure exerted upon the puncture opening is in some cases too high, and so there is a risk of circulation being stopped in the respective part of the body, and in some cases is too low so that there is a risk of haematoma formation.

It is therefore the object of the invention to specify an improved closure device for closing opened blood vessels.

This object is achieved by a closure device for closing opened blood vessels having the features of claim 1.

The closure device for closing opened blood vessels contains a closure element and a pressure body, the closure element being made of a material with elastic properties. According to the invention the closure device further contains a plurality of retaining strips which adjoin distal ends of the closure element, the retaining strips being provided at least in some areas with a reinforcing layer which is made of a material with non-elastic properties; and at least one indicator element which is connected to the closure element.

The closure device according to the invention for closing an opened blood vessel makes it possible, surprisingly easily, to apply a pressure that can be set individually by applying a tensile force to the closure device, to the puncture opening. The tensile force required for this purpose can be read off by means of the indicator element before the closure device is adhered. At least two indicator elements are provided in order to provide a better read-off. In other words, it is possible to draw a conclusion regarding the respective pressure applied to the puncture opening by means of the changeable shape or geometry of the indicator elements which is caused by the changeable tensile force applied to the closure device if the closure device is adhered in the state (when tensile force is applied).

The ability to read off the indicator elements extremely precisely is achieved surprisingly easily and reliably by the closure element having elastic properties and the retaining strips in turn having non-elastic properties. It should be mentioned here that extensions are respectively defined at the ends of the closure element as retaining strips. The user grasps the closure device on these retaining strips, for example between the index fingers and thumbs, and by extending the closure device exerts a tensile force upon the closure device. This tensile force is in turn absorbed almost exclusively by the material of the closure element which, according to the invention, has elastic properties. The closure device according to the invention allows easy and reliable handling. Moreover, the pressure applied to the puncture opening can be set individually and specifically to the application by reading off the indicator elements. Therefore, the correct pressure can always be set at the puncture point. Moreover, the structure of the closure device according to the invention is very simple, by means of which production costs are reduced.

Preferably, the closure element and the retaining strips are made in one piece. The closure device is provided here with retaining strips of an appropriate length. In addition to good handling, one should therefore also achieve sufficient adhesion to the skin in order to be able to establish the counter-pressure required to arrest the bleeding. The geometry of the closure device is not tied to this form. Other forms are also conceivable, such as for example circular patches. However, as mentioned above, the closure element must have an elastic property and the retaining strips must have a non-elastic property. In this way the required tensile force can be read off by means of the indicator elements so as to thus be able to apply an individually required pressure to the puncture opening.

Alternatively, the closure element and the retaining strips are preferably made in a number of pieces. In this embodiment the closure element made of an elastic material and the plurality of retaining strips made of a non-elastic material can be connected to one another by appropriate connection methods.

Preferably, the closure element and/or the pressure body and/or the indicator element are made in one piece. By forming these elements from just one material high cost savings can be achieved. Moreover, large numbers of items can be produced in a short time.

Preferably, the at least one indicator element is made in relation to the closure element as a recess or highlighted area which adopts a changeable geometry as the closure element is extended further by applying a tensile force to the latter. In this way, in response to a desired pressure which is applied to the puncture opening, a tensile force is applied to the ends of the closure device until the indicator elements adopt a (defined) geometry necessary for this purpose. As soon as the user visually reads off or identifies the geometry of the indicator elements defined for this purpose, the correct tensile force is applied and the closure device extended by this tensile force can be applied to the patient's skin. In this way it is advantageously possible to apply an individual pressure to the puncture opening by simple means.

Preferably, in an unstressed state of the closure element, the at least one indicator element is respectively formed in the geometry of an oval shape which, as the closure element is extended further, changes into an increasingly circular form and, as it is extended further, changes into an increasingly oval shape. By means of the configuration of the indicator elements in an oval shape particularly reliable setting of the respectively required tensile force applied to the closure device is made possible by visual inspection of the changeable shape. Depending on the medical indication a changed geometry is set. By extending the closure device the initially oval shape of the indicator elements becomes increasingly round. By extending the closure device beyond this extended state the geometry of the indicator elements changes from the round form into an oval form. By reading off the changeable form the required tensile force can be defined simply and reliably.

Preferably, the closure device also contains a liquid-absorbent element which is disposed around the pressure body on the peripheral side. This liquid-absorbent element soaks up any blood possibly passing out of the needle. From a hygienic point of view, the closure device also has advantages in comparison to known and established solutions. The closure device is provided as a sterile one-way product in order to also rule out cross-infections in particular.

Preferably, the liquid-absorbent element contains an active agent with pain-relieving and/or haemostatic and/or antiallergenic active agents. In this way good compatibility is even guaranteed when the closure device is applied to the patient for a long period of time.

Preferably, the reinforcing layer is respectively formed in the region of the retaining strips, on one side of the closure device on which the pressure body is provided. By means of this reinforcing layer the required non-elastic properties are imparted to the retaining strips. It is to be understood here that a material with non-elastic properties is to be understood as a material that does not allow any extension in the longitudinal or in the transverse direction. As can be understood from the above description of the field of application, deformability of the material is provided which allows the retaining strips to be nestled against and adhered even to non-level body areas of the person.

Preferably, the reinforcing layer is connected to the region of the retaining strips by means of an adhesive on one side respectively and is provided with a skin adhesive layer on the other side. With the aid of the skin adhesive layer the closure device is fixed on the patient's skin by means of adhesive force such that the pressure body thus reliably closes the puncture opening.

Preferably, the skin adhesive layer is covered by a removable protective film. In this way the skin adhesive layer is protected and so a long-lasting adhesive force of the skin adhesive layer can be guaranteed. One only needs to remove the protective film from the skin adhesive layer before applying the closure device to the surface of the patient's skin.

Preferably, the closure element and/or the pressure body and/or the retaining strips and/or the indicator element are made of silicone, natural rubber, synthetic rubber, gum, latex, hydrogel, polymeric plastic or a combination of these materials. As described above, the materials of the closure element have a high degree of elasticity and have a desirably high reset force. In particular, silicone is a preferred material for the closure device due to the outstanding medical suitability. A further advantage of choosing silicone is that the exceptionally high extensibility provided is limited and stabilised in the two-dimensional expansibility. This is necessary in order to be able to apply the individually settable counter-pressure required to arrest bleeding.

Preferably, the closure element and/or the pressure body and/or the retaining strips and/or the indicator element and/or the reinforcing layer are transparent or almost transparent. In this way the application of the closure device as a puncture closure at the correct position is facilitated.

Preferably, the retaining strips are permeable to water vapour. In this way the carrying property of the closure device is improved.

Preferably, the retaining strips are perforated. Water vapour can be discharged quickly through the perforations, and this improves the carrying property of the closure device.

In the following the closure device according to the invention is described in detail by means of the drawings. The drawings show as follows:

FIG. 1 is a longitudinal section through a closure device which can be adhered in a defined position;

FIG. 2 is a view of the closure device according to FIG. 1;

FIG. 3 is a diagrammatic sketch showing application of the closure device; and

FIGS. 4a-c are diagrammatic views of the closure device with different extensions.

FIG. 1 shows as an example a longitudinal section, not to scale, through a closure device 1 which can be adhered in a defined position, and FIG. 2 shows for this purpose a view from below of the closure device 1 according to FIG. 1. The closure device 1 contains a closure element 2 and a pressure body 3 which, in this example, is made in one piece of a material with elastic properties, for example silicone. The pressure body 3 is an approximately lens-shaped structure and serves to apply a compressive force in order to close a puncture opening (not shown).

Furthermore, two retaining strips 4 are provided as extensions of the closure elements 2 at distal ends of the latter. The retaining strips 4 have non-elastic properties. For example, the retaining strips 4 can be made in one piece with the closure element 2. In order to impart the non-elastic properties to the retaining strips 4 a reinforcing layer 5, for example, is applied to the retaining strips 4. The reinforcing layer 5 is in turn made of a material with non-elastic properties. The reinforcing layer 5 can be adhered onto the retaining strips 4 by means of a conventional (technical) adhesive 6. In a case where the retaining strips 4 are produced from silicone it may be necessary to prepare the contact surface appropriately with the adhesive 6. In order to improve the lasting contact of silicone with the adhesive used 6 a surface treatment such as e.g. a plasma, corona, wet chemical or other treatment can therefore be provided.

Alternatively, the common material from which the closure element 2 and the retaining strips 4 are formed in one piece can be processed, for example by specific curing processing sequences, such that only the material in the region of the retaining strips 4 does not allow any extension, while the remaining regions can be extended further.

Although not shown in the drawing, the retaining strips 4 and the closure element 2 are made of different materials which are connected to one another. In this example the closure element 2 can be made of silicone and the retaining strips 4 can be made of polyethylene (PE).

A skin adhesive layer 7 for adhesion to the skin is provided on the lower side of the reinforcing layer 5. The skin adhesive layer 7 preferably has skin adhesive-specific properties. Finally, the skin adhesive layer 7 can additionally also be provided with a removable protective film (not shown).

Furthermore, in the region of the closure element 2 the closure device 1 contains two indicator elements 8 which are connected to the closure element 2. In one embodiment the indicator elements 8 are made in one piece with the closure element 2. The indicator elements 8 are arranged here on both sides of the pressure body 3, preferably spaced equally. The indicator elements 8 are designed to take on a changeable geometry when extended further by applying a tensile force to the closure device 1 (and so to the closure element 2), which changeability is in relation to the extension and so to the tensile force.

The closure element 2, the pressure body 3, the retaining strips 4, the indicator elements 8 and the reinforcing layer 5 are preferably made to be transparent or almost transparent in order to facilitate the application of the closure device 1 which can be adhered in a defined position.

As already mentioned, the closure element 2, the pressure body 3, the retaining strips 4 and the indicator elements 8 are made of a one-piece silicone material. In addition to exceptional extensibility, silicone also has a desirably high reset force. However, other materials such as e.g. natural rubber, synthetic rubber, gum, latex, hydrogel, polymeric plastic or a combination of these materials can also be used. Alternatively, the retaining strips can be produced from polyethylene (PE).

In normal cases (for example following an examination and/or treatment etc.) the pressure body 3 is in direct contact with the skin. Therefore, appropriate medical properties, especially as regards compatibility with the skin and body, are required for the materials used.

By means of FIG. 3 the use of the closure device 1 which can be adhered in a defined position in order to close an opened vessel is described.

Before a needle or cannula 9 is withdrawn from the puncture point, the corresponding point of the body is cleaned and disinfected in the conventional manner. Next the protective films (not shown in the figures) are removed from both retaining strips of the closure device 1. The closure device 1 is then applied and fixed to the respective point of the body by means of the retaining strips such that later, upon full application of the closure device 1, the pressure body 3 comes to lie precisely over the puncture point, as indicated by the arrow in FIG. 3. Next the needle or cannula 9 is removed. Specified as areas of application for the closure device 1 are: dialysis, before removing the fistula needle; general medicine, before removing the indwelling cannula; radiology, before removing the catheter; minimally invasive surgery, before removing the endoscope and/or other investigative equipment.

In FIGS. 4a-c diagrammatic views of the closure device 1 with different extensions of the latter are shown. In this embodiment the indicator elements 8 are made in one piece with the closure element 2. The indicator elements 8 can, for example, being made as a recess or highlighted area in relation to the closure element. In a further example the indicator elements 8 can be made by removing material or applying material to just the contour of the indicator elements 8. More than two indicator elements 8 can also be provided.

In the original, unstressed state of the closure device 1 (see FIG. 4a) the indicator elements 8 are designed with the geometry of an oval shape. The centre of the indicator elements 8 should respectively come to lie in a section of the closure element 2 in the centre between the pressure body 3 and the projection to the retaining strip 4. Forms different to the oval shape are also conceivable for the indicator elements 8.

By extending the closure device 1 by respectively applying a tensile force F1 to the retaining strips 4 it is only the length of this element that is extended due to the elastic property of the material of the closure element 2. By means of this extension the indicator elements 8 are also extended with the result that the oval form of the latter increasingly changes into a circular shape, as shown in FIG. 4b.

By increasing the tensile forces of F1 to F2, the circular shape changes into an oval shape, as shown in FIG. 4c. By observing the respective form of the indicator elements 8 the user is capable of drawing a conclusion regarding the respective tensile force. In response to this the user is therefore capable of defining the pressure which the pressure body 3 of a closure device 1 thus extended applies to the puncture point as soon as the closure device 1 extended in this way is adhered to the patient's skin. The pressure can thus be defined quickly, easily and specifically to the application.

1. A closure device (1) for closing an opened blood vessel, comprising a closure element (2) and a pressure body (3), the closure element (2) being made of a material with elastic properties; characterised by a plurality of retaining strips (4) which adjoin distal ends of the closure element (2), the retaining strips (4) being provided at least in some areas with a reinforcing layer (5) which is made of a material with non-elastic properties; and at least one indicator element (8) which is connected to the closure element (2).

2. The closure device (1) according to claim 1, characterised in that the closure element (2) and the retaining strips (4) are made in one piece.

3. The closure device (1) according to claim 1, characterised in that the closure element (2) and the retaining strips (4) are made in a number of pieces.

4. The closure device (1) according to claim 1, characterised in that the closure element and/or the pressure body (3) and/or the at least one indicator element (8) are made in one piece.

5. The closure device (1) according to claim 1, characterised in that the at least one indicator element (8) is made in relation to the closure element (2) as a recess or highlighted area which adopts a changed geometry as the closure element (2) is extended further by applying a tensile force to the latter.

6. The closure device (1) according to claim 5, characterised in that, in an unstressed state of the closure element (2), the at least one indicator element (8) is respectively formed in the geometry of an oval shape which, as the closure element (2) is extended further, changes into an increasingly circular form and, as it is extended further, changes into an increasingly oval shape.

7. The closure device (1) according to claim 1, further characterised by a liquid-absorbent element which is disposed around the pressure body (3) on the peripheral side.

8. The closure device (1) according to claim 7, characterised in that the liquid-absorbent element contains an active agent with pain-relieving and/or haemostatic and/or antiallergenic active agents.

9. The closure device (1) according to claim 1, characterised in that the reinforcing layer (5) is respectively formed in the region of the retaining strips (4), on one side of the closure device (1) on which the pressure body (3) is provided.

10. The closure device (1) according to claim 9, characterised in that the reinforcing layer (5) is connected to the region of the retaining strips (4) by means of an adhesive (6) on one side respectively and is provided with a skin adhesive layer (7) on the other side.

11. The closure device (1) according to claim 10, characterised in that the skin adhesive layer (7) is covered by a removable protective film.

12. The closure device (1) according to claim 1, characterised in that the closure element (2) and/or the pressure body (3) and/or the retaining strips (4) and/or the at least one indicator element (7) are made of silicone, natural rubber, synthetic rubber, gum, latex, hydrogel, polymeric plastic or a combination of these materials.

13. The closure device (1) according to claim 1, characterised in that the closure element (2) and/or the pressure body (3) and/or the retaining strips (4) and/or the at least one indicator element (8) and/or the reinforcing layer (5) are transparent or almost transparent.

14. The closure device (1) according to claim 1, characterised in that the retaining strips (4) are permeable to water vapour.

15. The closure device (1) according to claim 1, characterised in that the retaining strips (4) are perforated.
